SEQUENCE LISTING (9 sequences)

SEQ ID NO: 1 - DNA, 843 bp, Homo sapiens
  CDS (1)..(840)
atgtgtagtgccttccatagggctgagtctgggaccgagctccttgcccgacttgaaggtagaagttccttgaaagaaatagaaccaaatctgtttgctgatgaagattcacctgtgcatggtgatattcttgaatttcatggcccagaaggaacaggaaaaacagaaatgctttatcacctaacagcacgatgtatacttcccaaatcagaaggtggcctggaagtagaagtcttatttattgatacagattaccactttgatatgctccggctagttacaattcttgagcacagactatcccaaagctctgaagaaataatcaaatactgcctgggaagattttttttggtgtactgcagtagtagcacccacttacttcttacactttactcactagaaagtatgttttgtagtcacccatctctctgccttttgattttggatagcctgtcagctttttactggatagaccgcgtcaatggaggagaaagtgtgaacttacaggagtctactctgaggaaatgttctcagtgcttagagaagcttgtaaatgactatcgcctggttctttttgcaacgacacaaactataatgcagaaagcctcgagctcatcagaagaaccttctcatgcctctcgacgactgtgtgatgtggacatagactacagaccttatctctgtaaggcatggcagcaactggtgaagcacaggatgtttttctccaaacaagatgattctcaaagcagcaaccaattttcattagtttcacgttgtttaaaaagtaacagtttaaaaaaacatttttttattattggagaaagtggggttgaattttgttga

SEQ ID NO: 2 - protein, 280 aa, Homo sapiens
MCSAFHRAESGTELLARLEGRSSLKEIEPNLFADEDSPVHGDILEFHGPEGTGKTEMLYHLTARCILPKSEGGLEVEVLFIDTDYHFDMLRLVTILEHRLSQSSEEIIKYCLGRFFLVYCSSSTHLLLTLYSLESMFCSHPSLCLLILDSLSAFYWIDRVNGGESVNLQESTLRKCSQCLEKLVNDYRLVLFATTQTIMQKASSSSEEPSHASRRLCDVDIDYRPYLCKAWQQLVKHRMFFSKQDDSQSSNQFSLVSRCLKSNSLKKHFFIIGESGVEFC

SEQ ID NO: 3 - DNA, 987 bp, Homo sapiens
  CDS (1)..(984)
atgggcgtgctcagggtcggactgtgccctggccttaccgaggagatgatccagcttctcaggagccacaggatcaagacagtggtggacctggtttctgcagacctggaagaggtagctcagaaatgtggcttgtcttacaaggccctggttgccctgaggcgggtgctgctggctcagttctcggctttccccgtgaatggcgctgatctctacgaggaactgaagacctccactgccatcctgtccactggcattggcagtcttgataaactgcttgatgctggtctctatactggagaagtgactgaaattgtaggaggcccaggtagcggcaaaactcaggtatgtctctgtatggcagcaaatgtggcccatggcctgcagcaaaacgtcctatatgtagattccaatggagggctgacagcttcccgcctcctccagctgcttcaggctaaaacccaggatgaggaggaacaggcagaagctctccggaggatccaggtggtgcatgcatttgacatcttccagatgctggatgtgctgcaggagctccgaggcactgtggcccagcaggtgactggttcttcaggaactgtgaaggtggtggttgtggactcggtcactgcggtggtttccccacttctgggaggtcagcagagggaaggcttggccttgatgatgcagctggcccgagagctgaagaccctggcccgggaccttggcatggcagtggtggtgaccaaccacataactcgagacagggacagcgggaggctcaaacctgccctcggacgctcctggagctttgtgcccagcactcggattctcctggacaccatcgagggagcaggagcatcaggcggccggcgcatggcgtgtctggccaaatcttcccgacagccaacaggtttccaggagatggtagacattgggacctgggggacctcagagcagagtgccacattacagggtgatcagacatga

SEQ ID NO: 4 - protein, 328 aa, Homo sapiens
MGVLRVGLCPGLTEEMIQLLRSHRIKTVVDLVSADLEEVAQKCGLSYKALVALRRVLLAQFSAFPVNGADLYEELKTSTAILSTGIGSLDKLLDAGLYTGEVTEIVGGPGSGKTQVCLCMAANVAHGLQQNVLYVDSNGGLTASRLLQLLQAKTQDEEEQAEALRRIQVVHAFDIFQMLDVLQELRGTVAQQVTGSSGTVKVVVVDSVTAVVSPLLGGQQREGLALMMQLARELKTLARDLGMAVVVTNHITRDRDSGRLKPALGRSWSFVPSTRILLDTIEGAGASGGRRMACLAKSSRQPTGFQEMVDIGTWGTSEQSATLQGDQT

SEQ ID NO: 5 - DNA, 34 bp, Artificial Sequence
  Synthetic DNA
gcatatgtgtagtgccttccatagggctgagtct

SEQ ID NO: 6 - DNA, 40 bp, Artificial Sequence
  Synthetic DNA
gggatccttaacaaaattcaaccccactttctccaataat

SEQ ID NO: 7 - DNA, 35 bp, Artificial Sequence
  Synthetic DNA
ggcatatgggcgtgctcagggtcggactgtgccct

SEQ ID NO: 8 - DNA, 38 bp, Artificial Sequence
  Synthetic DNA
ggcatatgttatgtctgatcaccctgtaatgtggcact

SEQ ID NO: 9 - DNA, 120 bp, Artificial Sequence
  Synthetic DNA
atttcttcatttcatgctagacagaagaattctcagtaacttctttgtgctgtgtgtattcaactcacagagtggaacgtccctttgcacagagcagatttgaaacactctttttgtagt

(19) United States
(12) Patent Application Publication
(10) Pub. No.: US 2003/0049818 A1
(43) Pub. Date: Mar. 13, 2003

(54) Polypeptide complex having DNA recombination activity

(76) Inventors: Hitoshi Kuruymizaka, Yokohama-shi (JP); Shigeyuki Yokoyama, Yokohama-shi (JP); Takehiko Shibata, Wako-shi (JP)

Correspondence Address: KNOBBE MARTENS OLSON & BEAR LLP, 2040 MAIN STREET, FOURTEENTH FLOOR, IRVINE, CA, 92614, US

(21) Appl. No.: 10/164,433

(22) Filed: Jun. 6, 2002

(30) Foreign Application Priority Data
Jun. 6, 2001 (JP) 2001-171705

(51) Int. Cl. G01N 33/53; C07H 21/04; C12N 9/00; C12P 21/02; C12N 5/06

(52) U.S. Cl. 435/183; 435/69.1; 435/320.1; 435/325; 435/7.1; 536/23.2

(57) ABSTRACT

A complex having homologous-pairing activity, produced by expressing a DNA encoding Xrcc2 and a DNA encoding Rad51D and recovering a complex of Xrcc2 and Rad51D; a vector comprising a DNA encoding Xrcc2 and a DNA encoding Rad51D; and a transformant having been transformed to enhance expressions of a DNA encoding Xrcc2 and a DNA encoding Rad51D.

### BACKGROUND OF THE INVENTION

The present invention relates to a polypeptide complex having homologous-pairing activity, to a method for producing the complex, to a vector usable for the production of the complex and to a transformant having an enhanced expression amount of the complex.

In cells, the genetic DNA ordinarily suffers damages. In particular, double-strand breaks in DNA that frequently occur by ionizing radiation or chemotherapeutics induce at disordered interchromosomal DNA recombination, which results in the occurrence of chromosomal aberration so that the cells lose their ability of ordered growth control. The double-strand breaks in chromosomal DNA are also induced by an error at the time of DNA replication. Such double-strand breaks in DNA remain unrepaired and accumulate in cells deficient in DNA recombination between sister chromosomes (homologous recombination), thereby giving serious damages to the chromosome.

Hitherto, human Rad51 (hereinafter, sometimes referred to also as “HsRad51”) has been reported as a human enzyme involved in the repair through homologous recombination. HsRad51 is a human homologue of Escherichia coli RecA protein, and the enzyme universally occurs in organisms ranging from bacteria to humans. The protein binds to a single-stranded DNA region exposed at the position of double-strand breaks in DNA and finds out the same nucleotide sequence as that of the region from its sister chromatid to perform DNA recombination reaction (FIG. 1). This enables cells to perform accurate repair of the site of the double-strand breaks in DNA without a difference of even a single base. This activity is referred to as homologous-paring activity.

In recent years, the existence of Rad51 family genes (Xrcc2, Xrcc3, Rad51B, Rad51C, and Rad51D) having about 20% homology with the HsRad51 gene has been reported to exist in humans (FIG. 2). Among those, human Xrcc2 and Xrcc3 genes, which complement sensitivity of hamster mutant cells cisplatin known to be a chemotherapeutic to cancers or to radiation, are believed to be important to the repair of the double-strand breaks in DNA due to such an exogenous factor. Further, because the phenotypes of cells deficient in Xrcc2 and Xrcc3 genes bear a striking resemblance to those of cells suffering from a genetic disease that is prone to cancers, it is suggested the possibility that these genes by themselves are involved in the suppression of cancers.

### SUMMARY OF THE INVENTION

An object of the present invention is to provide a substance that exhibits homologous-pairing activity. Further, an object of the present invention is to provide a method for producing the substance. Another object of the present invention is to provide a vector for use in the production method. Still another object of the present invention is to provide a transformant having high homologous-pairing activity.

The inventors of the present invention have made biochemical analysis of the Rad51 family proteins for the purpose of helping one understand the mechanism of repair of double-strand breaks in DNA in humans. The present invention has been achieved based on the findings from this analysis.

That is, according to the present invention, there is provided a complex which comprises a polypeptide represented by one of the following (A) and (B) and a polypeptide represented by one of the following (C) and (D) (hereinafter also referred to as a complex of the present invention):

(A) a polypeptide having the amino acid sequence of SEQ ID NO:2;

(B) a polypeptide having an amino acid sequence comprising substitution, deletion, insertion, addition, or inversion of one or several amino acids in the amino acid sequence of SEQ ID NO:2, which can constitute an enzyme having homologous-pairing activity by forming a complex with a polypeptide having the amino acid sequence of SEQ ID NO:4;

(C) a polypeptide having the amino acid sequence of SEQ ID NO:4; and

(D) a polypeptide having an amino acid sequence comprising substitution, deletion, insertion, addition, or inversion of one or several amino acids in the amino acid sequence of SEQ ID NO:4, which can constitute an enzyme having homologous-pairing activity by forming a complex with a polypeptide having the amino acid sequence of SEQ ID NO:2.

The complex of the present invention is preferably a complex comprising a polypeptide having the amino acid sequence of SEQ ID NO:2 and a polypeptide having the amino acid sequence of SEQ ID NO:4.

Further, according to the present invention, there is provided a method for producing the complex. That is, according to the present invention, there is provided a method for producing a complex which comprises a polypeptide represented by one of the following (A) and (B) and a polypeptide represented by one of the following (C) and (D), the method comprising expressing, in one system, a DNA encoding the polypeptide represented by one of the following (A) and (B) and a DNA encoding the polypeptide represented by one of the following (C) and (D) to produce a complex of the polypeptides and then recovering the produced complex (hereinafter also referred to as a production method of the present invention):

(A) a polypeptide having the amino acid sequence of SEQ ID NO:2;

(B) a polypeptide having an amino acid sequence comprising substitution, deletion, insertion, addition, or inversion of one or several amino acids in the amino acid sequence of SEQ ID NO:2, which can constitute an enzyme having homologous-pairing activity by forming a complex with a polypeptide having the amino acid sequence of SEQ ID NO:4;

(C) a polypeptide having the amino acid sequence of SEQ ID NO:4; and

(D) a polypeptide having an amino acid sequence comprising substitution, deletion, insertion, addition, or inversion of one or several amino acids in the amino acid sequence of SEQ ID NO:4, which can constitute an enzyme having homologous-pairing activity by forming a complex with a polypeptide having the amino acid sequence of SEQ ID NO:2.

The production method of the present invention is preferably a method for producing a complex which comprises a polypeptide having the amino acid sequence of SEQ ID NO:2 and a polypeptide having the amino acid sequence of SEQ ID NO:4, the method comprising expressing, in one system, a DNA encoding the polypeptide having the amino acid sequence of SEQ ID NO:2 and a DNA encoding the polypeptide having the amino acid sequence of SEQ ID NO:4 to produce a complex of the polypeptides and then recovering the produced complex.

According to the present invention, there is further provided a vector which may be used for the production method of the present invention. That is, according to the present invention, there is provided a vector comprising a first DNA which encodes a polypeptide represented by one of the following (A) and (B) and a second DNA which encodes a polypeptide represented by one of the following (C) and (D) (hereinafter also referred to as a vector of the present invention):

(A) a polypeptide having the amino acid sequence of SEQ ID NO:2;

(B) a polypeptide having an amino acid sequence comprising substitution, deletion, insertion, addition, or inversion of one or several amino acids in the amino acid sequence of SEQ ID NO:2, which can constitute an enzyme having homologous-pairing activity by forming a complex with a polypeptide having the amino acid sequence of SEQ ID NO:4;

(C) a polypeptide having the amino acid sequence of SEQ ID NO:4; and

(D) a polypeptide having an amino acid sequence comprising substitution, deletion, insertion, addition, or inversion of one or several amino acids in the amino acid sequence of SEQ ID NO:4, which can constitute an enzyme having homologous-pairing activity by forming a complex with a polypeptide having the amino acid sequence of SEQ ID NO:2.

The vector of the present invention is preferably a vector comprising a first DNA which encodes a polypeptide having the amino acid sequence of SEQ ID NO:2 and a second DNA which encodes a polypeptide having the amino acid sequence of SEQ ID NO:4.

Further, the vector of the present invention is preferably a vector comprising a first DNA represented by one of the following (a) and (b) and a second DNA represented by one of the following (c) and (d):

(a) a DNA having the nucleotide sequence of SEQ ID NO:1;

(b) a DNA which hybridizes with a DNA having the nucleotide sequence of SEQ ID NO:1 under stringent conditions and encodes a polypeptide which can constitute an enzyme having homologous-pairing activity by forming a complex with a polypeptide having the amino acid sequence of SEQ ID NO:4;

(c) a DNA having the nucleotide sequence of SEQ ID NO:3; and

(d) a DNA which hybridizes with a DNA having the nucleotide sequence of SEQ ID NO:3 under stringent conditions and encodes a polypeptide which can constitute an enzyme having homologous-pairing activity by forming a complex with a polypeptide having the amino acid sequence of SEQ ID NO:2.

More preferably, the vector of the present invention is a vector comprising a first DNA having the nucleotide sequence of SEQ ID NO:1 and a second DNA having the nucleotide sequence of SEQ ID NO:3.

It is preferable that in the vector of the present invention, the first DNA and the second DNA are arranged in tandem in the same direction and transcriptions thereof are controlled by promoters of the same kind.

Still further, according to the present invention, there is provided a transformant which has been transformed to enhance expressions of the DNAs encoding the polypeptides that constitute the complex of the present invention. That is, according to the present invention, there is provided a transformant which has been transformed to enhance expressions of a DNA which encodes a polypeptide represented by one of the following (A) and (B) and a DNA which encodes a polypeptide represented by one of the following (C) and (D) (hereinafter also referred to as a transformant of the present invention):

(A) a polypeptide having the amino acid sequence of SEQ ID NO:2;

(B) a polypeptide having an amino acid sequence comprising substitution, deletion, insertion, addition, or inversion of one or several amino acids in the amino acid sequence of SEQ ID NO:2, which can constitute an enzyme having homologous-pairing activity by forming a complex with a polypeptide having the amino acid sequence of SEQ ID NO:4;

(C) a polypeptide having the amino acid sequence of SEQ ID NO:4; and

(D) a polypeptide having an amino acid sequence comprising substitution, deletion, insertion, addition, or inversion of one or several amino acids in the amino acid sequence of SEQ ID NO:4, which can constitute an enzyme having homologous-pairing activity by forming a complex with a polypeptide having the amino acid sequence of SEQ ID NO:2.

The transformant of the present invention is preferably a transformant which has been transformed to enhance expressions of a DNA which encodes a polypeptide having the amino acid sequence of SEQ ID NO:2, and a DNA which encodes a polypeptide having the amino acid sequence of SEQ ID NO:4.

Still further, the transformant of the present invention is preferably a transformant which has been transformed by introducing a DNA having the nucleotide sequence represented by one of the following (a) and (b) and a DNA having the nucleotide sequence represented by one of the following (c) and (d):

(a) the nucleotide sequence of SEQ ID NO:1;

(b) the nucleotide sequence of a DNA which hybridizes with a DNA having the nucleotide sequence of SEQ ID NO:1 under stringent conditions and encodes a polypeptide which can constitute an enzyme having homologous-pairing activity by forming a complex with a polypeptide having the amino acid sequence of SEQ ID NO:4;

(c) the nucleotide sequence of SEQ ID NO:3; and

(d) the nucleotide sequence of a DNA which hybridizes with a DNA having the nucleotide sequence of SEQ ID NO:3 under stringent conditions and encodes a polypeptide which can constitute an enzyme having homologous-pairing activity by forming a complex with a polypeptide having the amino acid sequence of SEQ ID NO:2.

More preferably, the transformant of the present invention is a transformant that has been transformed with the vector of the present invention.

Note that the terms “polypeptide” and “protein” referred to herein are used as synonyms to each other.

The present invention provides a polypeptide complex having high homologous-pairing activity and a method for producing the same. Further, the present invention provides a vector for use in the production method and a transformant exhibiting high homologous-pairing activity.

### BRIEF EXPLANATION OF THE DRAWINGS

FIG. 1 shows an explanatory diagram illustrating homologous-pairing reaction;

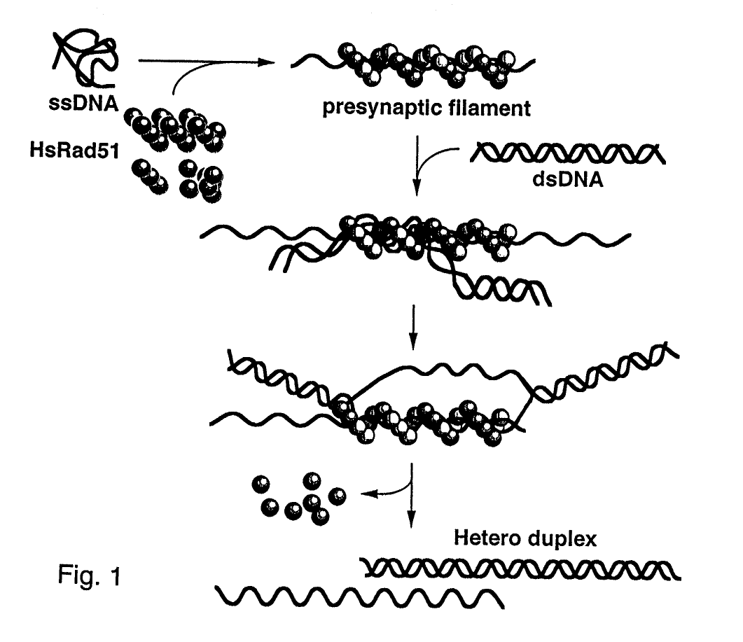

FIG. 2 shows comparison of the structures of Rad51 family proteins with each other;

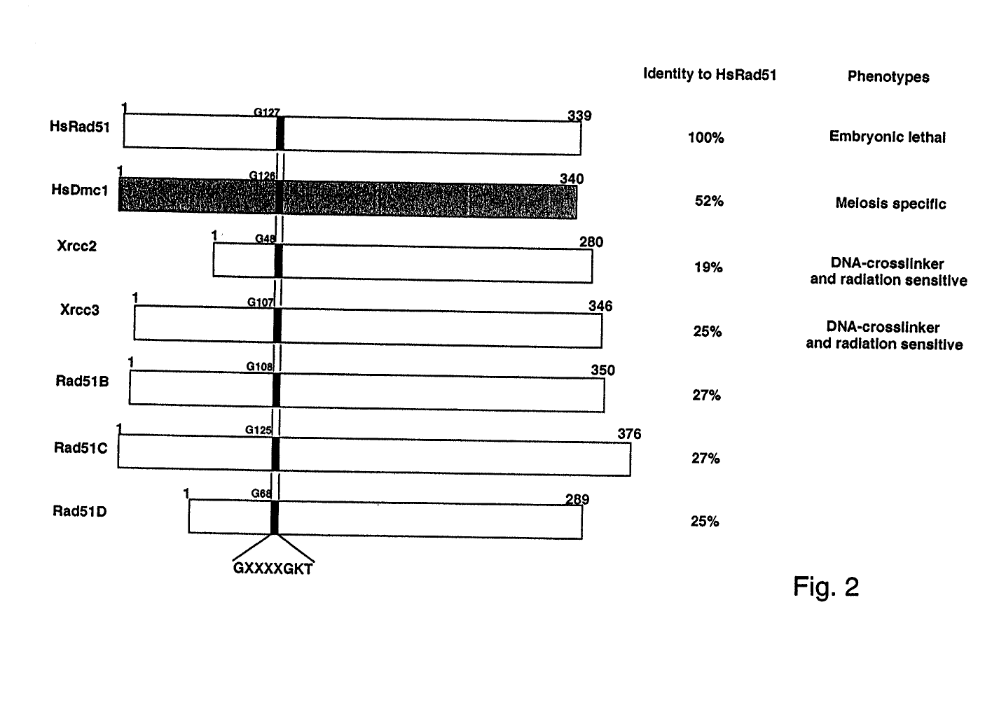

FIG. 3 shows an electrophoretogram of fractions of Ni column chromatography in the purification of Xrcc2·Rad51D complex;

FIG. 4 shows an electrophoretogram of a purified Xrcc2·Rad51D complex;

FIG. 5 shows electrophoretograms illustrating DNA binding activity of Xrcc2·Rad51D complex;

FIG. 6 shows an electrophoretogram illustrating homologous-pairing activity; and

FIG. 7 shows an electron micrograph of a complex between Xrcc2·Rad51D complex and DNA. DE

### DETAILED DESCRIPTION OF THE INVENTION

<1> Complex of the invention

A first polypeptide that constitutes the complex of the present invention includes a polypeptide having the amino acid sequence of SEQ ID NO:2 and a second polypeptide that constitutes the complex of the present invention includes a polypeptide having the amino acid sequence of SEQ ID NO:4.

The polypeptides having the amino acid sequences of SEQ ID NO:2 and SEQ ID NO:4, respectively, are Rad15 family proteins known as Xrcc2 and Rad51D.

The complex of the present invention has homologous-pairing activity. The term “homologous-pairing activity” as used herein means the activity of forming a pair between two homologous DNA strands and can be evaluated based on the activity of forming a D-loop from a single-stranded DNA and a double-stranded DNA homologous thereto. Specifically, the homologous-pairing activity can be measured by the method described in Examples referred to later.

Generally, it is known that there may be differences in an amino acid sequence of a protein, that give no influence on the function of the protein. This is based on the existence of amino acids similar in the properties, such as leucine and isoleucine, and the presence of a portion in a protein that does not participate in any function in the three-dimensional structure of the protein.

Therefore, the first polypeptide that constitutes the complex of the present invention is not limited to the polypeptide having the amino acid sequence of SEQ ID NO:2 but may be any one of the polypeptides having amino acid sequences comprising substitution, deletion, insertion, addition, or inversion of one or several amino acids in the amino acid sequence of SEQ ID NO:2, which can constitute an enzyme having homologous-pairing activity by forming a complex with the polypeptide having the amino acid sequence of SEQ ID NO:4. The polypeptides including that having the amino acid sequence of SEQ ID NO:2 are also collectively called Xrcc2. Xrcc2 is preferably a polypeptide having the amino acid sequence of SEQ ID NO:2.

Also, the second polypeptide that constitutes the complex of the present invention is not limited to the polypeptide having the amino acid sequence of SEQ ID NO:4 but may be any one of the polypeptides having amino acid sequences comprising substitution, deletion, insertion, addition, or inversion of one or several amino acids in the amino acid sequence of SEQ ID NO:4, which can constitute an enzyme having homologous-pairing activity by forming a complex with the polypeptide having the amino acid sequence of SEQ ID NO:2. The polypeptides including that having the amino acid sequence of SEQ ID NO:4 are also collectively called Rad51D. Rad51D is preferably a polypeptide having the amino acid sequence of SEQ ID NO:4.

The terms “having homologous-pairing activity” usually means having an activity equivalent to the homologous-pairing activity of the complex constituted by the polypeptide having the amino acid sequence of SEQ ID NO:2 and the polypeptide having the amino acid sequence of SEQ ID NO:4 when evaluated by the homologous-pairing activity measurement method described in the Examples described later (usually, showing a D-loop amount of 5% or more based on the double-stranded DNA as a substrate when the band of D-loop in an electrophoretogram is evaluated by densitometry).

Note that the term “several amino acids” as used herein refers to usually 100 amino acids or less although such may vary depending on the kind of amino acid and its position in the polypeptide.

The complex of the present invention can be obtained by the production method of the present invention described below.

<2> Production method of the present invention

The production method of the present invention is a method for producing a complex between Xrcc2 and Rad51D, comprising expressing an Xrcc2-encoding DNA and an Rad51D-encoding DNA in one system to produce a complex of polypeptides and then recovering the produced complex.

A nucleotide sequence of the Xrcc2-encoding DNA is known (Molecular Cell, 1, 783-793 (1998)). The Xrcc2-encoding DNA can be obtained based on the known nucleotide sequence by the PCR method in which a human cDNA library is used as a template. The cDNA library includes the one derived human testis. The primer used in the PCR method includes primers having the nucleotide sequences of SEQ ID NO:5 and SEQ ID NO:6, respectively.

The Xrcc2-encoding DNA includes the nucleotide sequence of SEQ ID NO:1. Also, it includes a DNA that hybridizes with a DNA having the nucleotide sequence of SEQ ID NO:1 under stringent conditions and encodes a polypeptide that can constitute an enzyme having homologous-pairing activity by forming a complex with a polypeptide having the amino acid sequence of SEQ ID NO:4. Such a DNA that encodes a polypeptide equivalent to the polypeptide having the amino acid sequence of SEQ ID NO:2 can be obtained, for example, from an allogenic mutant due to artificial mutation with treatment with a known mutagen or by spontaneous mutation.

The term “stringent conditions” as used herein refers to the conditions under which DNAs having high homology specifically hybridize, for example, the condition of performing hybridization at 42° C. in 5× SSC, 5× Denhardt's solution and 0.1% SDS and washing with 0.1× SSC and 0.1% SDS at 55° C.

A nucleotide sequence of Rad51D-encoding DNA is known (Biochemical and Biophysical Research Communications, 257, 156-162 (1999)). The Rad51D-encoding DNA can be obtained based on the known nucleotide sequence by the PCR method in which a human cDNA library is used as a template. The cDNA library includes the one derived from human testis. The primer used in the PCR method includes primers having the nucleotide sequences of SEQ ID NO:7 and SEQ ID NO:8, respectively.

The Rad51D-encoding DNA includes the nucleotide sequence of SEQ ID NO:3. Also, it includes a DNA that hybridizes with a DNA having the nucleotide sequence of SEQ ID NO:3 under stringent conditions and encodes a polypeptide that can constitute an enzyme having homologous-pairing activity by forming a complex with a polypeptide having the amino acid sequence of SEQ ID NO:2. Such a DNA that encodes a polypeptide equivalent to the polypeptide having the amino acid sequence of SEQ ID NO:4 can be obtained, for example, from an allogenic mutant due to artificial mutation with treatment with a known mutagen or by spontaneous mutation.

The system in which the Xrcc2-encoding DNA and Rad51D-encoding DNA are expressed is not particularly limited as far as the polypeptides produced by the expression of both DNAs produce a complex. The system may be either a cell in which both DNAs are introduced or a cell-free transcription and translation system. The cell may be either a prokaryotic cell or an eukaryotic cell and includes, for example, cells of Escherichia coli, insects (for example, Sf9) and yeast. The cell-free transcription and translation system includes, for example, reticulocyte lysate and Escherichia coli extract solutions.

The Xrcc2-encoding DNA and Rad51D-encoding DNA may be operatively connected to a regulatory sequence that is compatible with the system, such as a promoter as necessary.

The Xrcc2-encoding DNA and Rad51D-encoding DNA may be introduced so that they are contained either in separate transcription units or in one transcription unit. In other words, two vectors having one and the other of the DNAs, respectively, or a single vector having both DNAs in separate transcription units may be used. The term “transcription unit” as used herein means a transcription region controlled by one promoter. It is preferred that the Xrcc2-encoding DNA and Rad51D-encoding DNA are introduced so that Xrcc2 and Rad51D can be generated in equimolar amounts. In this respect, it is preferred that both genes are arranged in tandem in the same direction and transcriptions thereof are controlled by promoters of the same kind. The term “arranged in tandem” means the arrangement so close to each other that no difference in expression due to position effect and the like can be observed.

For the cleavage, ligation, introduction into a cell and the like of DNA, the known method described in detail in Molecular Cloning, 2nd edition, Cold Spring Harbor Press (1989) and the like can be used.

In a case where cells are used as the system, the produced complex can be recovered by desrupting the cells by a known method and isolating the aimed complex from the system. Because the property of the complex has been elucidated in the present invention, the isolation of the aimed complex may be performed by a combination of known means used for the purification of proteins by using as an index the elucidated property. For example, the aimed complex can be purified by column chromatography using an Ni column (in the case where a His tag is used) and ion exchange resin.

Note that the term “encoding” is used herein as including the case where a DNA has a sequence such as an intron or an addition sequence as far as after transcription and/or translation, RNA and/or polypeptide undergoes processing to form the aimed polypeptide.

<3> Vector of the present invention

The vector of the present invention has a first DNA encoding Xrcc2 and a second DNA encoding Rad51D. The vector can be used in the production method of the present invention.

The first DNA encoding Xrcc2 includes DNA having the nucleotide sequence of SEQ ID NO:1 and DNA encoding a polypeptide equivalent to the polypeptide having the amino acid sequence of SEQ ID NO:2, with the DNA having the nucleotide sequence of SEQ ID NO:1 being preferred.

The second DNA encoding Rad51D includes DNA having the nucleotide sequence of SEQ ID NO:3 and DNA encoding a polypeptide equivalent to the polypeptide having the amino acid sequence of SEQ ID NO:4, with the DNA having the nucleotide sequence of SEQ ID NO:3 being preferred.

It is preferred that the first DNA and the second DNA are arranged in tandem in the same direction and transcriptions thereof are controlled by promoters of the same kind.

The vector of the present invention can be obtained by inserting an Xrcc2-encoding DNA and an Rad51D-encoding DNA into a vector. The vector into which the Xrcc2-encoding DNA and the Rad51D-encoding DNA are to be inserted may be a known vector, examples of which include pET15b and pGEX. The promoter that controls the transcription of both DNAs may be a known promoter, examples of which include T7, tac, lac, and R.

For the cleavage, ligation and the like of DNA, the known method described in detail in Molecular Cloning, 2nd edition, Cold Spring Harbor Press (1989) and the like can be used.

The Xrcc2-encoding DNA and the Rad51D-encoding DNA can be obtained in the same manner as described for the production method of the present invention as described above.

<4> Transformant of the present invention

The transformant of the present invention is a transformant which has been transformed to enhance expressions of an Xrcc2-encoding DNA and an Rad51D-encoding DNA.

The Xrcc2-encoding DNA includes DNA having the nucleotide sequence of SEQ ID NO:1 and DNA encoding a polypeptide equivalent to the polypeptide having the amino acid sequence of SEQ ID NO:2, with the DNA having the nucleotide sequence of SEQ ID NO:1 being preferred.

The Rad51D-encoding DNA includes DNA having the nucleotide sequence of SEQ ID NO:3 and DNA encoding a polypeptide equivalent to the polypeptide having the amino acid sequence of SEQ ID NO:4, with the DNA having the nucleotide sequence of SEQ ID NO:3 being preferred.

The term “transformed to enhance” as used herein means to have increased in production amount of a polypeptide that the DNA encodes, as compared with the cell before transformation. The method of enhancing the expression of a polypeptide includes a method of increasing the copy number of DNA (gene) encoding the aimed polypeptide in the cell, a method of increasing the expression amount of the gene and the like. The method of increasing the copy number of the gene includes a method of introducing a multicopy vector into the cell. The method of increasing the expression amount of the gene includes a method of promoting transcription, for example, by modifying a promoter and a method of promoting translation, for example, by modifying a recognition site of a translational regulation factor. The gene, the expression amount of which has been increased, may be retained in the cell as extrachromosomal nucleic acid or integrated into the chromosome by homologous recombination.

The vector to be introduced into a cell is preferably the vector of the present invention.

The host to be transformed is not particularly limited and may be either a prokaryote cell or an eukaryotic cell and includes, for example, cells of Escherichia coli, insects (for example, Sf9) and yeast.

For the cleavage, ligation, introduction into a cell and the like of DNA, the known method described in detail in Molecular Cloning, 2nd edition, Cold Spring Harbor Press (1989) and the like can be used.

The Xrcc2-encoding DNA and the Rad51D-encoding DNA can be obtained in the same manner as described for the production method of the present invention as described above.

### EXAMPLES

Hereinafter, the present invention will be described in more detail by way of examples. Example 1: Xrcc2/Rad51D coexpression vector

(1) Cloning of human Xrcc2 and Rad51D genes

(a) Xrcc2

PCR was performed using the primers described below and also the cDNA described below as a template under the following PCR conditions. Primer:

XRCC2-Nde: 5′ GCATATGTGTAGTGCCTTCCATAGGGCTGAGTCT 3′ (SEQ ID NO:5)

XRCC2-Bam: 5′ GGGATCCTTAACAAAATTCAACCCCACTTTCTCCAATAAT 3′ (SEQ ID NO:6)

cDNA: Marathon-Ready CDNA (human testis, Clontech)

PCR Conditions:

Step 1=94° C., 1 min., 1 cycle;

Step 2=94° C., 30 sec.,

Step 3=72° C., 2 min., Steps 2-3: 5 cycles;

Step 4=94° C., 30 sec.,

Step 5=70° C., 2 min., Steps 4-5: 5 cycles;

Step 6=94° C., 20 sec.,

Step 7=68° C., 2 min., Steps 6-7: 40 cycles.

The obtained DNA fragment was inserted into PGEM-T Easy vector (Promega) to confirm the sequence. Thereafter, NdeI-BamHI-cleaved DNA fragment was inserted into the NdeI-BamHI site of pET15b vector (Novagen) to obtain a pET15b-Xrcc2 vector.

(b) Rad51D

PCR was performed using the primers described below and also the CDNA described below as a template under the following PCR conditions. Primer:

51D-FW: 5′ GGCATATGGGCGTGCTCAGGGTCGGACTGTGCCCT 3′ (SEQ ID NO:7)

51D-RV: 5′ GGCATATGTTATGTCTGATCACCCTGTAATGTGGCACT 3′ (SEQ ID NO:8)

cDNA: Marathon-Ready cDNA (human testis, Clontech)

PCR Conditions:

Step 1=94° C., 1 min., 1 cycle;

Step 2=94° C., 30 sec.,

Step 3=70° C., 2 min., Steps 2-3: 5 cycles;

Step 4=94° C., 30 sec.,

Step 5=66° C., 2 min., Steps 4-5: 5 cycles;

Step 6=94° C., 20 sec.,

Step 7=62° C., 2 min., Steps 6-7: 40 cycles.

The obtained DNA fragment was inserted into PGEM-T Easy vector (Promega) to confirm the sequence. Thereafter, NdeI-cleaved DNA fragment was inserted into the NdeI site of pET15b vector (Novagen) to obtain a pET15b-Rad51D vector.

(2) Construction of vector

(a) Xrcc2/Rad51D coexpression vector

The Bg1II-BaMHI fragment of pET15b-Xrcc2 vector was inserted into the Bg1II site of the pET15b-Rad51D vector prepared by the above-mentioned method. The vector contained Xrcc2 and Rad51D genes arranged in tandem in the same direction and had a T7 promoter upstream of each of the genes.

Example 2: Xrcc2·Rad51D complex

(1) Preparation of a complex

The coexpression vector prepared in Example 1 was introduced into E. coli JM109(DE3) cells and each gene product was over-expressed as a protein having a His₆ tag on the N-terminus. Specifically, the coexpression vector obtained in Example 1 was introduced into the E. coli JM109(DE3) cells together with the expression vector of arginine genes (ARG3 and ARG4). The cells were cultured at 30° C. to a turbidity of 0.4 to 0.8 (measured at 600 nm). After cooling to 18° C., IPTG (isopropyl thio-β-galactopyranoside) was added to the medium in a final concentration of 0.2 mM and the cells were cultured at 18° C. for 12 to 16 hours.

Then, the cells obtained from 10 liters of the culture broth were suspended in 30 ml of 20 mM Tris-HCl (pH 8.5) buffer containing 0.5 M NaCl and disrupted by an ultrasonic cell disrupter. The cell debris were removed by centrifugation (30000×g, 20 minutes) to obtain a supernatant (cell extraction). To the supernatant was added 4 ml of Ni-bound resin (ProBond, Invitrogen) and the mixture was left to stand for 1 hour. Then the resin was washed with 50 ml of 20 mM Tris-HCl (pH 8.5) buffer containing 0.5 M NaCl four times and subsequently with 50 ml of 20 mM Tris-HCl (pH 8.5) buffer containing 5 mM imidazole four times. After packing the resin in a column, elution was done with 100 ml of a linear gradient of 5-400 mM imidazole in 20 mM Tris-HCl (pH 8.5) buffer. The flow rate was 0.67 ml/min and the fraction volume was 2 ml. Each fraction was analyzed by 12% SDS-PAGE and Coomassie Blue staining and fractions in which the major bands were two bands of the polypeptides constituting the complex were collected. The collected fractions were applied to Mono-Q column (Amersham Pharmacia Biotech) equilibrated with 20 mM Tris-HCl (pH 8.0) buffer containing 10% glycerol. After washing it with the buffer used in the equilibration, elution was done with a linear gradient of 0-1.2 M NaCl in 20 mM Tris-HCl (pH 8.0) buffer containing 10% glycerol. The fraction volume was 0.5 ml. Each fraction was analyzed by 12% SDS-PAGE and Coomassie Blue staining and fractions in which the major bands were two bands of the polypeptides constituting the complex were collected. The collected fractions were dialyzed against 20 mM Tris-HCl (pH 7.5), 5 mM dithiothreitol and 10% glycerol to obtain a purified protein. The purified protein was stored in 20 mM Tris-HCl (pH 8.0), 5 mM dithiothreitol and 10% glycerol. The operations subsequent to the disruption of the cells were performed at 4° C.

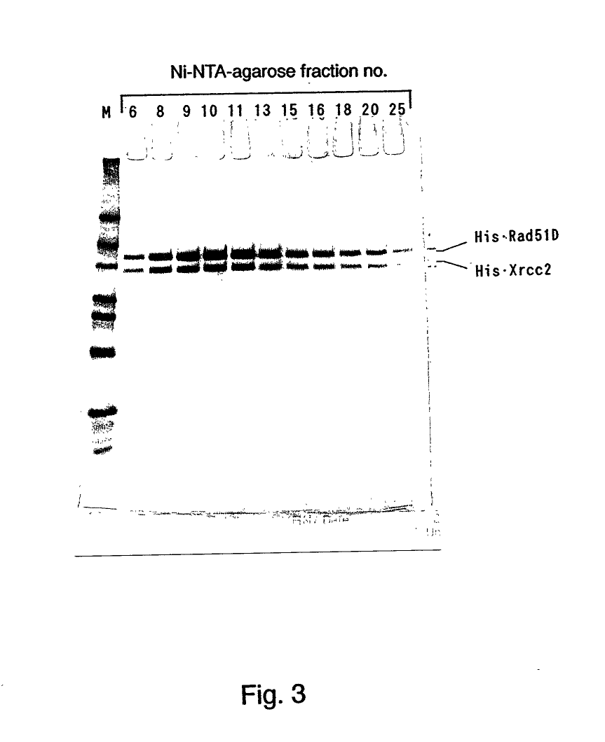

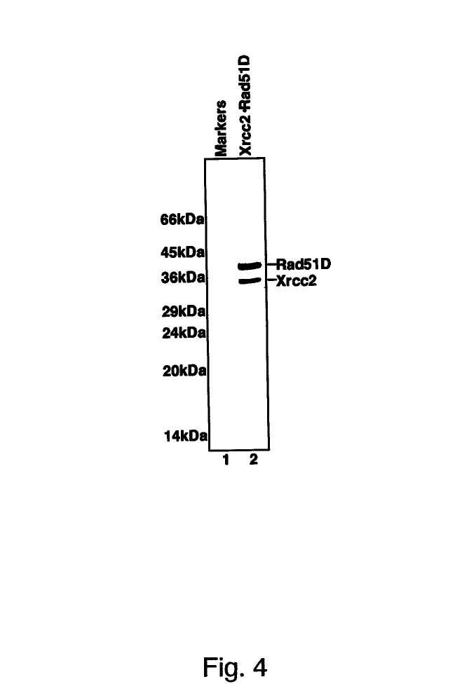

FIG. 3 shows the results of analyses by 12% SDS-PAGE and Coomassie Blue staining of eluted fractions when the cell extract in the Xrcc2/Rad51D coexpression system is subjected to Ni column chromatography. As shown in FIG. 3, Xrcc2 and Rad51D were co-eluted. The Xrcc2 and Rad51D eluted from the Ni column behaved coincidently in subsequent Mono-Q column chromatography, and further gel filtration column (Superdex 200HR, Amersham Pharmacia Biotech) chromatography. Therefore, it was confirmed that Xrcc2 formed a complex with Rad51D. FIG. 4 shows the results of analysis of the purified Xrcc2/Rad51d complex by 12% SDS-PAGE and Coomassie Blue staining.

(2) Measurement of homologous-pairing activity

Single-stranded and double-stranded DNA substrates were prepared as follows.

The 5′ end of a 120-mer oligonucleotide (5′-ATTTCTTCATTTCATGCTAGACAGAAGAATTCTCAGTAACTTCTTTGTGCTGTGTG TATTCAACTCACAGAGTGGAACGTCCCTTTGCACAGAGCAGATTTGAAACACTCTT TTTGTAGT-3′(SEQ ID NO:9) (Boehringer Mannheim) purified by HPLC was labeled by using T4 polynucleotide kinase (New England Biolabs) and [γ-³²P]ATP. The oligonucleotide thus prepared will be hereinafter referred to as ³²P-lableled single-stranded 120-mer oligonucleotide. The nucleotide sequence of the oligonucleotide was derived from a human α-satellite DNA clone.

A 198-base pair fragment of human α-satellite DNA was cloned in PGEM-T Easy vector (Promega). Plasmid DNA containing the human a-satellite DNA (pGsat4; 3216 base pairs) was purified by an ultracentrifugation method using a sucrose density gradient. The double-stranded DNA of Form I thus prepared will be hereinafter referred to also as “superhelical pGsat4 DNA”. Furthermore, a single-stranded cyclic DNA was prepared from E. coli having pGsat4 by using helper phage.

The concentration of DNA was expressed in terms of mole number.

(2-1) DNA binding activity of a complex Single-stranded pGsat4 DNA (20 μM) was mixed with the Xrcc2·Rad51D complex in 10 μl of a standard reaction buffer containing 50 mM Tris-HCl (pH 8.0), 2 mM ATP, 20 mM creatine phosphate, 1 mM dithiothreitol, 100 μg/ml BSA, 12 U/ml creatine phosphokinase, 2 mM MgCl₂, and 5% glycerol. The reaction mixture was incubated at 37° C. for 10 minutes and analyzed by 0.8% agarose gel electrophoresis in 0.5× TBE buffer. The electrophoresis was performed at 3 V/cm for 4 hours. DNA was detected by staining with ethidium bromide. Assays were similarly performed by using superhelical pGsat4 DNA (double-stranded) instead of the single-stranded pGsat 4 DNA.

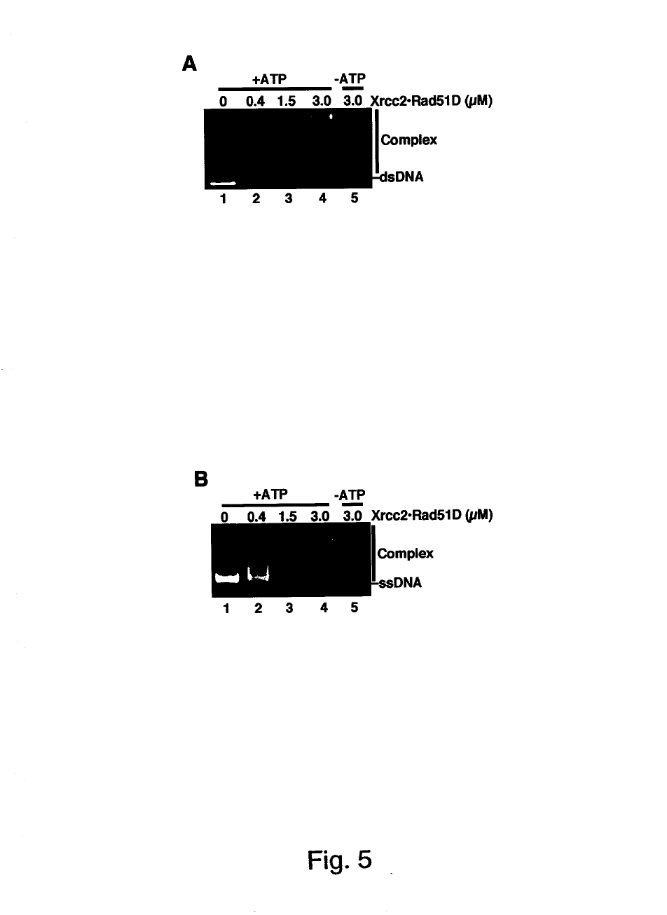

FIG. 5 shows the results. FIG. 5, A shows the results obtained with the double-stranded DNA (dsDNA) and FIG. 5, B shows the results with single-stranded DNA (ssDNA). The concentrations of proteins were 0 μM (Lane 1), 0.4 μM (Lane 2), 1.5 μM (Lane 3), 3.0 μM (Lane 4), and 3.0 μM (without ATP) (Lane 5).

As will be apparent from the results shown in FIG. 5, the Xrcc2·Rad51D complex bound to both of the single-stranded DNA and the double-stranded DNA.

(2-2) Homologous-pairing activity of the complex

The ³²P-labeled single-stranded 120-mer oligonucleotide (300 nM) was mixed with the Xrcc2·Rad51D complex in 10 μl of a standard reaction buffer containing 50 mM Tris-HCl (pH 8.0), 2 mM ATP, 20 mM creatine phosphate, 1 mM dithiothreitol, 100 μg/ml BSA, 12 U/ml creatine phosphokinase, and 2 mM MgCl₂. The reaction mixture was incubated at 37° C. for 10 minutes and then superhelical pGsat4 DNA (20 μM) was added thereto to start the reaction. After 20 minutes' incubation at 37° C., 0.5% SDS and proteinase K (700 μg/ml) were added to stop the reaction and the reaction mixture was incubated at 37° C. for 15 minutes to remove proteins. The product of homologous pairing was separated by 0.8% agarose gel electrophoresis in 0.5× TBE buffer. The amount of the ³²P-labeled single-stranded oligonucleotide incorporated in the D-loop was measured by using BAS2500 Image Analyzer (Fuji Photo Film).

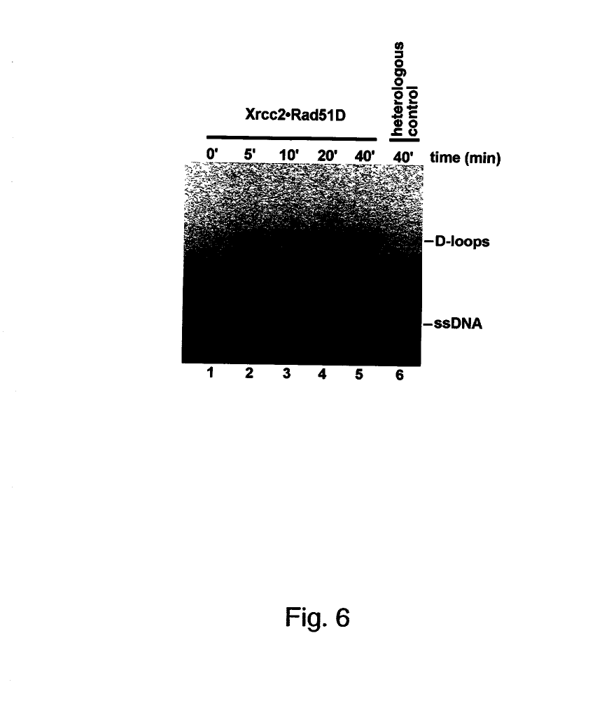

FIG. 6 shows the results. The amount of protein was 3 μM and the reaction time was 0 minute (Lane 1), 5 minutes (Lane 2), 10 minutes (Lane 3), 20 minutes (Lane 4), or 40 minutes (Lanes 5 and 6). Lane 6 was a control in which a heterologous double-stranded DNA was used.

In FIG. 6, the concentrations of the band designated by “D-loop” indicate occurrence of homologous pairing (D-loop formation) between the single-stranded DNA and the superhelical double-stranded DNA. As will be apparent from FIG. 6, increasing reaction time results in increased D-loop formation. Therefore, it can be understood that the complex has high homologous-pairing activity.

(3) Observation of the binding of the complex to DNA under a microscope

The single-stranded pGsat4 DNA (3 μM) was incubated together with 0.2 μM Xrcc2·Rad51D complex at 37° C. for 10 minutes in a buffer containing 20 mM Tris-HCl (pH 7.8), 10 mM dithiothreitol, 1 mM ATP, and 10% glycerol. The obtained complex was observed on JEM 2000FX electron microscope (JOEL). Note that all the complexes were visualized by negative staining with uranyl acetate.

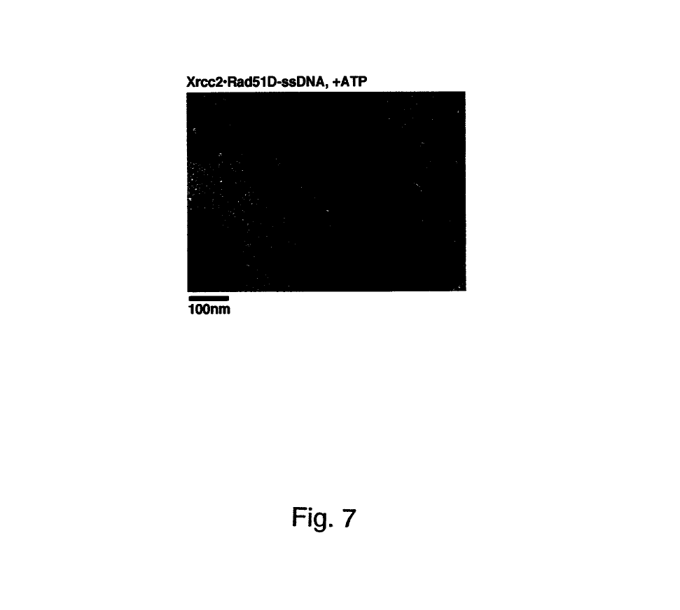

FIG. 7 shows the results obtained. The magnification bar in FIG. 7 represents 100 nm. It was observed that the Xrcc2·Rad51D complex formed a complex with the single-stranded DNA.

What is claimed is:

1. A complex comprising a polypeptide represented by one of the following (A) and (B) and a polypeptide represented by one of the following (C) and (D):
(A) a polypeptide having the amino acid sequence of SEQ ID NO:2;
(B) a polypeptide having an amino acid sequence comprising substitution, deletion, insertion, addition, or inversion of one or several amino acids in the amino acid sequence of SEQ ID NO:2, which can constitute an enzyme having homologous-pairing activity by forming a complex with a polypeptide having the amino acid sequence of SEQ ID NO:4;
(C) a polypeptide having the amino acid sequence of SEQ ID NO:4; and
(D) a polypeptide having an amino acid sequence comprising substitution, deletion, insertion, addition, or inversion of one or several amino acids in the amino acid sequence of SEQ ID NO:4, which can constitute an enzyme having homologous-pairing activity by forming a complex with a polypeptide having the amino acid sequence of SEQ ID NO:2.

2. A complex according to claim 1, which comprises a polypeptide having the amino acid sequence of SEQ ID NO:2 and a polypeptide having the amino acid sequence of SEQ ID NO:4.

3. A method for producing a complex which comprises a polypeptide represented by one of the following (A) and (B) and a polypeptide represented by one of the following (C) and (D), the method comprising expressing, in one system, a DNA encoding the polypeptide represented by one of the following (A) and (B) and a DNA encoding the polypeptide represented by one of the following (C) and (D) to produce a complex of the polypeptides and then recovering the produced complex:
(A) a polypeptide having the amino acid sequence of SEQ ID NO:2;
(B) a polypeptide having an amino acid sequence comprising substitution, deletion, insertion, addition, or inversion of one or several amino acids in the amino acid sequence of SEQ ID NO:2, which can constitute an enzyme having homologous-pairing activity by forming a complex with a polypeptide having the amino acid sequence of SEQ ID NO:4;
(C) a polypeptide having the amino acid sequence of SEQ ID NO:4; and
(D) a polypeptide having an amino acid sequence comprising substitution, deletion, insertion, addition, or inversion of one or several amino acids in the amino acid sequence of SEQ ID NO:4, which can constitute an enzyme having homologous-pairing activity by forming a complex with a polypeptide having the amino acid sequence of SEQ ID NO:2.

4. A method according to claim 3, which comprises expressing, in one system, a DNA encoding the polypeptide having the amino acid sequence of SEQ ID NO:2 and a DNA encoding the polypeptide having the amino acid sequence of SEQ ID NO:4 to produce a complex of the polypeptides and then recovering the produced complex.

5. A vector comprising a first DNA which encodes a polypeptide represented by one of the following (A) and (B) and a second DNA which encodes a polypeptide represented by one of the following (C) and (D):
(A) a polypeptide having the amino acid sequence of SEQ ID NO:2;
(B) a polypeptide having an amino acid sequence comprising substitution, deletion, insertion, addition, or inversion of one or several amino acids in the amino acid sequence of SEQ ID NO:2, which can constitute an enzyme having homologous-pairing activity by forming a complex with a polypeptide having the amino acid sequence of SEQ ID NO:4;
(C) a polypeptide having the amino acid sequence of SEQ ID NO:4; and
(D) a polypeptide having an amino acid sequence comprising substitution, deletion, insertion, addition, or inversion of one or several amino acids in the amino acid sequence of SEQ ID NO:4, which can constitute an enzyme having homologous-pairing activity by forming a complex with a polypeptide having the amino acid sequence of SEQ ID NO:2.

6. A vector according to claim 5, which comprises a first DNA which encodes a polypeptide having the amino acid sequence of SEQ ID NO:2 and a second DNA which encodes a polypeptide having the amino acid sequence of SEQ ID NO:4.

7. A vector comprising a first DNA represented by one of the following (a) and (b) and a second DNA represented by one of the following (c) and (d):
(a) a DNA having the nucleotide sequence of SEQ ID NO:1;
(b) a DNA which hybridizes with a DNA having the nucleotide sequence of SEQ ID NO:1 under stringent conditions and encodes a polypeptide which can constitute an enzyme having homologous-pairing activity by forming a complex with a polypeptide having the amino acid sequence of SEQ ID NO:4;
(c) a DNA having the nucleotide sequence of SEQ ID NO:3; and
(d) a DNA which hybridizes with a DNA having the nucleotide sequence of SEQ ID NO:3 under stringent conditions and encodes a polypeptide which can constitute an enzyme having homologous-pairing activity by forming a complex with a polypeptide having the amino acid sequence of SEQ ID NO:2.

8. A vector according to claim 7, which comprises a first DNA having the nucleotide sequence of SEQ ID NO:1 and a second DNA having the nucleotide sequence of SEQ ID NO:3.

9. A vector according to claim 5, wherein the first DNA and the second DNA are arranged in tandem in the same direction and transcriptions thereof are controlled by promoters of the same kind.

10. A vector according to claim 6, wherein the first DNA and the second DNA are arranged in tandem in the same direction and transcriptions thereof are controlled by promoters of the same kind.

11. A vector according to claim 7, wherein the first DNA and the second DNA are arranged in tandem in the same direction and transcriptions thereof are controlled by promoters of the same kind.

12. A vector according to claim 8, wherein the first DNA and the second DNA are arranged in tandem in the same direction and transcriptions thereof are controlled by promoters of the same kind.

13. A transformant which has been transformed to enhance expressions of a DNA which encodes a polypeptide represented by one of the following (A) and (B) and a DNA which encodes a polypeptide represented by one of the following (C) and (D):
(A) a polypeptide having the amino acid sequence of SEQ ID NO:2;
(B) a polypeptide having an amino acid sequence comprising substitution, deletion, insertion, addition, or inversion of one or several amino acids in the amino acid sequence of SEQ ID NO:2, which can constitute an enzyme having homologous-pairing activity by forming a complex with a polypeptide having the amino acid sequence of SEQ ID NO:4;
(C) a polypeptide having the amino acid sequence of SEQ ID NO:4; and
(D) a polypeptide having an amino acid sequence comprising substitution, deletion, insertion, addition, or inversion of one or several amino acids in the amino acid sequence of SEQ ID NO:4, which can constitute an enzyme having homologous-pairing activity by forming a complex with a polypeptide having the amino acid sequence of SEQ ID NO:2.

14. A transformant according to claim 13, which has been transformed to enhance expressions of a DNA which encodes a polypeptide having the amino acid sequence of SEQ ID NO:2 and a DNA which encodes a polypeptide having the amino acid sequence of SEQ ID NO:4.

15. A transformant according to claim 13, which has been transformed by introducing a DNA having the nucleotide sequence represented by one of the following (a) and (b) and a DNA having the nucleotide sequence represented by one of the following (c) and (d):
(a the nucleotide sequence of SEQ ID NO:1;
(b) the nucleotide sequence of a DNA which hybridizes with a DNA having the nucleotide sequence of SEQ ID NO:1 under stringent conditions and encodes a polypeptide which can constitute an enzyme having homologous-pairing activity by forming a complex with a polypeptide having the amino acid sequence of SEQ ID NO:4;
(c) the nucleotide sequence of SEQ ID NO:3; and
(d) the nucleotide sequence of a DNA which hybridizes with a DNA having the nucleotide sequence of SEQ ID NO:3 under stringent conditions and encodes a polypeptide which can constitute an enzyme having homologous-pairing activity by forming a complex with a polypeptide having the amino acid sequence of SEQ ID NO:2.

16. A transformant which has been transformed with the vector as defined in claim 5.

17. A transformant which has been transformed with the vector as defined in claim 6.

18. A transformant which has been transformed with the vector as defined in claim 7.

19. A transformant which has been transformed with the vector as defined in claim 8.

20. A transformant which has been transformed with the vector as defined in claim 9.
